(19) United States
(12) Patent Application Publication
(10) Pub. No.: US 2024/0156891 A1
(43) Pub. Date: May 16, 2024

(54) EXTRACTS OF SALVIA MILTIORRHIZA AND/OR PONCIRUS TRIFOLIATA FOR INHIBITING BODY WEIGHT GAIN

(71) Applicants: Taipei Medical University, Taipei City (TW); I-MEI FOODS CO., LTD, Taipei City (TW)

(72) Inventor: Heng LIN, Taipei City (TW)

(73) Assignees: Taipei Medical University, Taipei City (TW); I-MEI FOODS CO., LTD, Taipei City (TW)

(21) Appl. No.: 18/386,470

(22) Filed: Nov. 2, 2023

Related U.S. Application Data
(60) Provisional application No. 63/424,555, filed on Nov. 11, 2022.

(51) Int. Cl. A61K 36/752; A61K 36/537; A61K 36/9066; A61P 3/04

(52) U.S. Cl. CPC A61K 36/752; A61K 36/537; A61K 36/9066; A61P 3/04

(57) ABSTRACT

The present invention relates to an extract of Salvia miltiorrhiza and/or an extract of Poncirus trifoliata for inhibiting obesity. In particular, the extract of Salvia miltiorrhiza and/or the extract of Poncirus trifoliata is/are prepared by solvent or supercritical fluid extraction. Specifically, the extract of Salvia miltiorrhiza and/or the extract of Poncirus trifoliata is/are effective in inhibiting lipid accumulation in adipocytes and slowing down body weight gain of the subject. More specifically, the extract of Salvia miltiorrhiza and/or the extract of Poncirus trifoliata can be used in combination with an extract of Curcuma longa, which exhibit improved effects in inhibiting lipid accumulation in adipocytes and slowing down body weight gain of the subject.

### RELATED APPLICATIONS

This application claims the benefit of U.S. provisional application No. 63/424,555, filed Nov. 11, 2022 under 35 U.S.C. § 119, the entire content of which is incorporated herein by reference.

### TECHNOLOGY FIELD

The present invention relates to an extract of Salvia miltiorrhiza and/or an extract of Poncirus trifoliata for inhibiting obesity. In particular, the extract of Salvia miltiorrhiza and/or the extract of Poncirus trifoliata is/are prepared by solvent or supercritical fluid extraction. Specifically, the extract of Salvia miltiorrhiza and/or the extract of Poncirus trifoliata is/are effective in inhibiting lipid accumulation in adipocytes and slowing down body weight gain of the subject. More specifically, the extract of Salvia miltiorrhiza and/or the extract of Poncirus trifoliata can be used in combination with an extract of Curcuma longa, which exhibit improved effects in inhibiting lipid accumulation in adipocytes and slowing down body weight gain of the subject.

### BACKGROUND OF THE INVENTION

With economic development, environmental changes, stable food supply, and the influence of factors such as changes in human eating habits, insufficient physical activity, etc., most modern people face the problem of obesity. Obesity not only causes changes in appearance, but also positively correlates with many chronic diseases (e.g., diabetes, hypertension, heart disease, fatty liver, etc.) and endangers health. Therefore, it is necessary to develop compositions that effectively reduce fat accumulation in adipocytes and reduce weight gain so as to ameliorate obesity and prevent various metabolic syndromes resulted from obesity.

Common methods of suppressing obesity include lifestyle change, diet control, exercise, treatment by medicine, etc. However, due to busy work, it is difficult for modem people to change lifestyle and strictly implement diet control and regular exercise in order to lose weight, let alone achieving controlling effect on adipocytes. Moreover, unguided diet control and exercise would result in poor weight loss outcome when the intensity of which is insufficient, and easily cause bodily harm when they are excessive.

Regarding treatment by medicine, there are many medicines for weight loss on the market, the mechanisms of which are nothing more than reducing appetite, promoting gastrointestinal motility and causing diarrhea, and increasing basal metabolic rate. For example, Xenical® functions by binding with stomach and pancreatic lipases in the gastrointestinal tract, thereby inhibiting the activity of lipases, causing fat in foods failing to be broken down into fatty acids and losing the opportunity to be absorbed. Although Xenical® can block fat absorption from foods, it accompanies with other risk factors, e.g., hypertension, diabetes, hyperlipidemia, etc. Therefore, the use of medicines for weight loss actually causes many adverse side effects. There is still a need to develop a means to achieve effective body weight control and inhibit obesity.

### SUMMARY OF THE INVENTION

The present invention is at least based on the finding that extracts of the commonly consumed Chinese herbal medicine, Salvia miltiorrhiza and/or Poncirus trifoliata, are effective in inhibiting lipid accumulation in adipocytes and slowing down body weight gain of a subject. It is also found in the present invention that an extract of Salvia miltiorrhiza and/or an extract of Poncirus trifoliata in combination with an extract of Curcuma longa exhibit improved effects in inhibiting lipid accumulation in adipocytes and slowing down body weight gain of the subject. It is further found that an extract of Salvia miltiorrhiza and/or an extract of Poncirus trifoliata optionally in combination with an extract of Curcuma longa can improve liver function.

Therefore, the preset invention provides a method for inhibiting body weight gain in a subject in need thereof, comprising administering to the subject an effective amount of an extract of Salvia miltiorrhiza and/or an extract of Poncirus trifoliata, and optionally an extract of Curcuma longa.

In some embodiments, the extract of Salvia miltiorrhiza is prepared by extracting Salvia miltiorrhiza with a first solvent, the extract of Poncirus trifoliata is prepared by extracting Poncirus trifoliata with a second solvent and/or the extract of Curcuma longa is prepared by extracting Curcuma longa with a third solvent

In some embodiments, the extract of Salvia miltiorrhiza, the extract of Poncirus trifoliata and/or the extract of Curcuma longa is/are obtained by supercritical fluid extraction.

In some embodiments, the first solvent for extracting Salvia miltiorrhiza, the second solvent for extracting Poncirus trifoliata and/or the extract of Curcuma longa is/are an alcohol. Examples of the alcohol include but are not limited to methanol, ethanol, propanol and butanol.

In some embodiments, the supercritical fluid extraction utilizes carbon dioxide as solvent.

In some embodiments, the extract of Salvia miltiorrhiza and/or the extract of Poncirus trifoliata, and optionally the extract of Curcuma longa is/are administrated in an amount effective in inhibiting lipid accumulation in adipocytes.

In some embodiments, the extract of Salvia miltiorrhiza and/or the extract of Poncirus trifoliata, and optionally the extract of Curcuma longa is/are administrated in an amount effective in treating or avoiding obesity of the subject.

In some embodiments, the extract of Salvia miltiorrhiza and the extract of Poncirus trifoliata are administered to the subject.

In some embodiments, the extract of Salvia miltiorrhiza and the extract of Poncirus trifoliata are administered in amounts effective in inhibiting body weight gain in the subject to an extent greater than administration of the extract of Salvia miltiorrhiza or the extract of Poncirus trifoliata alone does.

In some embodiments, the extract of Salvia miltiorrhiza, the extract of Poncirus trifoliata and the extract of Curcuma longa are administered to the subject.

In some embodiments, the extract of Salvia miltiorrhiza and/or the extract of Poncirus trifoliata, and optionally the extract of Curcuma longa is/are administrated in amounts effective in improving liver function of the subject.

In some embodiments, the extract of Salvia miltiorrhiza and/or the extract of Poncirus trifoliata, and optionally the extract of Curcuma longa is/are administrated in amounts effective in reducing glutamic oxaloacetic transaminase (GOT) levels and/or glutamic pyruvic transaminase (GPT) levels in the subject.

The present invention also provides a composition for inhibiting body weight gain in a subject in need thereof comprising an extract of Salvia miltiorrhiza and/or an extract of Poncirus trifoliata, and optionally an extract of Curcuma longa as described herein and a physiologically acceptable carrier, wherein the extract of Salvia miltiorrhiza and/or the extract of Poncirus trifoliata, and optionally the extract of Curcuma longa are present in amounts effective in inhibiting body weight gain in a subject in need thereof. The present invention also provides a kit comprising (i) an extract of Salvia miltiorrhiza; and/or (ii) an extract of Poncirus trifoliata, and optionally (iii) an extract of Curcuma longa. Also provided is use of an extract of Salvia miltiorrhiza and/or an extract of Poncirus trifoliata, and optionally an extract of Curcuma longa as described herein for manufacturing a composition for inhibiting body weight gain in a subject in need thereof.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIGS. 1A and 1B show the effects of inhibiting the formation of lipid droplets in adipocytes by the extracts of the present invention. A supercritical carbon dioxide extract of Salvia miltiorrhiza (SME-CO₂) (1.2 μg/ml) and an ethanol extract of Poncirus trifoliata (PTE-EtOH) (10 μg/ml) each exhibit inhibitory effects on the formation of lipid droplets in adipocytes while a combination of SME-CO₂ (1.2 μg/ml) and PTE-EtOH (10 μg/ml) shows a better inhibitory effect on the formation of lipid droplets in adipocytes, as compared to that achieved by SME-CO₂ (1.2 μg/ml) or PTE-EtOH (10 ng/ml) alone (FIG. 1A). An ethanol extract of Salvia miltiorrhiza SME-EtOH (25 ng/ml) and PTE-EtOH (10 ng/ml) each exhibit inhibitory effect on the formation of lipid droplets in adipocytes while a combination of SME-EtOH (25 μg/ml) and PTE-EtOH (10 μg/ml) shows a better inhibitory effect on the formation of lipid droplets in adipocytes, as compared to that achieved by SME-EtOH (25 μg/ml) or PTE-EtOH (10 ng/ml) alone (FIG. 1B). Statistical comparisons were performed using unpaired t test. Data are presented as mean±SEM. **p<0.01, ***p<0.001 compared with the control group.

FIG. 2 shows the weight change of high-fat diet (HFD) induced obese mice administered through intraperitoneal injection with SME-EtOH (600 mg/kg/week), SME-CO₂ (15 mg/kg/week) or PTE-EtOH (75 mg/kg/week) for 6 weeks after 4-week HFD supply. Weight gain of HFD-induced obese mice administered with SME-EtOH (600 mg/kg/week), SME-CO₂ (15 mg/kg/week) or PTE-EtOH (75 mg/kg/week) was slower than that of the control group. Statistical comparisons were performed using two-way ANOVA. Data are presented as mean±SEM. ***p<0.001 compared with the control group.

FIG. 3 shows the weight change of high-fat diet (HFD) induced obese mice administered through intraperitoneal injection with SME-EtOH (300 mg/kg/week), EEP (50 mg/kg/week) or a combination thereof for 7 weeks after 4-week HFD supply. The combination of SME-EtOH (300 mg/kg/week) and PTE-EtOH (50 mg/kg/week) significantly reduces the weight gain. Statistical comparisons were performed using two-way ANOVA. Data are presented as mean±SEM. **p<0.01, ***p<0.001 compared with the control group.

FIG. 4 shows the weight change of high-fat diet (HFD) induced obese mice administered through intraperitoneal injection with SME-CO₂ (5 mg/kg/week), PTE-EtOH (50 mg/kg/week) or a combination thereof for 7 weeks after 4-week HFD supply. The combination of SME-CO₂ (5 mg/kg/week) and PTE-EtOH (50 mg/kg/week) significantly reduces the weight gain. Statistical comparisons were performed using two-way ANOVA. Data are presented as mean±SEM. *p<0.05, ***p<0.001 compared with the control group.

FIG. 5 shows the effects of inhibiting the formation of lipid droplets in adipocytes by the extracts of the present invention. A combination of SME-EtOH (5 μg/ml) and PTE-EtOH (5 μg/ml) and a combination of SME-EtOH (5 μg/ml) and CLE-EtOH (1 μg/ml) exhibit synergistic inhibitory effects on the formation of lipid droplets in adipocytes while individual SME-EtOH (5 μg/ml), PTE-EtOH (5 μg/ml) and CLE-EtOH (1 μg/ml) do not exhibit such inhibitory effects. A combination of SME-EtOH (5 μg/ml), PTE-EtOH (5 μg/ml) and CLE-EtOH (1 μg/ml) shows a more synergistic inhibitory effect on the formation of lipid droplets in adipocytes, as compared to that achieved by a combination of SME-EtOH (5 μg/ml) and PTE-EtOH (5 μg/ml) and that achieved by a combination of SME-EtOH (5 μg/ml and CLE-EtOH (1 μg/ml). Statistical comparisons were performed using unpaired t test. Data are presented as mean±SEM. **p<0.01, ***p<0.001. compared with the indicated group.

FIG. 6 shows the weight change of high-fat diet (HFD) induced obese mice administered through oral with SME-EtOH (600 mg/kg/week), SME-EtOH (600 mg/kg/week) plus PTE-EtOH (75 mg/kg/week), and SME-EtOH (600 mg/kg/week), PTE-EtOH (75 mg/kg/week) plus CLE-EtOH (50 mg/kg/week) for 14 weeks after 4-week HFD supply. SME-EtOH (600 mg/kg/week) plus PTE-EtOH (75 mg/kg/week) and also SME-EtOH (600 mg/kg/week), PTE-EtOH (75 mg/kg/week) plus CLE-EtOH (50 mg/kg/week) significantly reduce the weight gain compared to control group at 14 weeks. Also, SME-EtOH (600 mg/kg/week), PTE-EtOH (75 mg/kg/week) plus CLE-EtOH (50 mg/kg/week) is more effective for weight loss than SME-EtOH (600 mg/kg/week) plus PTE-EtOH (75 mg/kg/week). Statistical comparisons were performed using two-way ANOVA. Data are presented as mean±SEM. *p<0.05, ***p<0.0 compared with the indicated group.

### DETAILED DESCRIPTION OF THE INVENTION

The following description is merely intended to illustrate various embodiments of the invention. As such, specific embodiments or modifications discussed herein are not to be construed as limitations to the scope of the invention. It will be apparent to one skilled in the art that various changes or equivalents may be made without departing from the scope of the invention.

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “comprise” or “comprising” is generally used in the sense of include/including which means permitting the presence of one or more features, ingredients or components. The term “comprise” or “comprising” encompasses the term “consists” or “consisting of.”

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value.

As used herein, the terms “subject,” “individual” and “patient” refer to any mammalian subject for whom diagnosis, prognosis, treatment, or therapy is desired, particularly humans. Other subjects may include cattle, dogs, cats, guinea pigs, rabbits, rats, mice, horses, and so on.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to slowing the progression or reversing the progression of a disease. In yet another embodiment, “treat”, “treating” or “treatment” refers to preventing or delaying the development or progression of the disease or disorder.

The term “effective amount” as used herein refers to the amount of the active ingredient that imparts the desired biological effect in the individual or cell to be treated. The effective amount may vary depending on various reasons, such as the route and frequency of administration, the weight and species of the individual receiving the active ingredient, and the purpose of administration. Based on the content disclosed herein, the methods established, and their own experience, those skilled in the art can determine the dosage in each case.

Salvia miltiorrhiza, also named Danshen or red sage, is a traditional Chinese medicinal herb that belongs to the genus Salvia of the Lamiaceae family. The roots and rhizomes of Salvia miltiorrhiza are collected and dried to obtain Radix Salviae Miltiorrhizae as a Chinese medicine that is useful in promoting blood flow and treating vascular disease as known in the art.

Poncirus trifoliata is a member of the Rutaceae family of plants. The fruits of Poncirus trifoliata are widely used as a traditional Chinese medicine for treating allergic inflammation as known in the art.

Curcuma longa is a member of the Zingiberaceae family of plants. The rhizomes of Curcuma longa (turmeric) are known to have various effects, such as antibacterial, anti-inflammatory, and anti-allergic properties, and are used for various medicinal purposes.

The term “extract” as used herein refers to a product or preparation obtained by extraction of a substance. In general, an extract is a solution or concentrated preparation obtained by soaking or mixing a substance to be extracted with a solvent. There are various particular extraction processes known in the art, including but not limited to, maceration, percolation, repercolation, digestion, counter-current extraction, turbo-extraction, or supercritical fluid extraction. Typically, an extract can be prepared from a fresh plant or a dried and/or powdered plant sample. The ratio of a substance to be extracted to a solvent can be about 1:1 to about 1:100 (w/v, g/ml), particularly about 1:1 to about 1:50 (w/v, g/ml), more particularly about 1:3 to about 1:30 (w/v, g/ml), even more particularly about 1:5 to 1:20 (w/v, g/ml). Extraction may be conducted at a proper temperature, for example at a temperature from about 20° C. to about 100° C., for example, from about 25° C. to about 80° C., from about 25° C. to about 50° C., or from about 35° C. to about 80° C. One may select proper types of solvents or adjust their concentrations to achieve proper polarity so as to perform extraction as needed. In certain embodiments, extraction can be performed by the following process: milling or slicing the plant materials, e.g., roots, rhizomes or fruits and soaking the milled or sliced materials in a desired solvent for a period of time sufficient to extract the active agents from the materials. The period of time for extraction may be 15 minutes or more, for example, 15 minutes to 5 hours, 20 minutes to 4 hours, 25 minutes to 3 hours, or 30 minutes to 2 hours. The soaking step may be performed with stirring or ultrasonication (ultrasonic-assisted extraction) to enhance the efficiency of extraction. The solid debris (residues) are removed by filtration; the resultant liquid (supernatant) is collected; the soaking or mixing step may be repeated as needed; the resultant liquid (supernatant) is combined, and further concentrated (e.g., under reduced pressure, by nitrogen blowing, or by centrifugal vacuum), purified or isolated. In some cases, the product can be dried to produce a powder extract. In certain embodiments, supercritical fluid extraction is performed including the following process: placing the material to be extracted in a container and filling it with supercritical fluid until the pressure and temperature rise by a considerable amount, which help the fluid to achieve a very high solubility capacity to extract the ingredients out from the material. Typically, supercritical fluid extraction is conducted with CO₂ as the supercritical fluid (solvent), using ethanol and methanol as co-solvents.

Proper types of solvents for extraction include water, ethers (e.g. ether, tetrahydrofuran (THF), 1,4-dioxane, methyl tert-butyl ether, and glycol dimethyl ether), alcohols (e.g. methanol, ethanol, propanol, butanol, n-propanol, isopropanol, n-butanol, 1-pentanol, 2-butoxyethanol), alkanes (e.g. n-pentane, n-hexane and n-heptane), ketones (e.g. acetone), and acids (e.g. acetic acid). Solvents may be removed by concentration through nitrogen blowing, reduced pressure or centrifugal vacuum, for example, to obtain a final product of extraction free of solvent. A proper solvent for extraction may have an n-octanol/water partition coefficient (log K_{ow}) ranging from −0.9 to 4.0. Some examples of solvents and their typical log K_{ow} values are shown in Table 1 below.

TABLE 1
Examples of solvents and their log K_{ow} values

| Solvent | log K_{ow} |
|---|---|
| methanol | −0.82~−0.66 |
| ethanol | −0.31~−0.32 |
| n-butanol | 0.84 |
| isopropyl alcohol | 0.05 |
| acetone | −0.24 |
| ethyl acetate | 0.66~0.73 |
| n-hexane | 3.17~3.94 |

In some embodiments, alcohols are used as the solvent for extraction. In certain embodiments, alcohols at a concentration of 50% to 95% are used for extraction. In certain embodiments, alcohols at a concentration of 90% or more such as 90% to 95% are used for extraction.

For the purpose of delivery and absorption, an effective amount of the active ingredient can be formulated with a physiologically acceptable carrier to form a composition in an appropriate form. Depending on the mode of administration, the composition of the present invention may contain about 0.1% to about 100% by weight of the active ingredient, wherein the percentage is calculated based on the total weight of the composition.

The term “physiologically acceptable” as used herein means that the carrier is compatible with the active ingredient of the composition, and preferably can stabilize the active ingredient and is safe for the individual receiving it. The carrier can be a diluent, carrier, excipient, or matrix for the active ingredient. Some examples of suitable excipients include glucose, sucrose, lactose, sorbitol, mannitol, starch, calcium phosphate, Arabic gum, alginate, tragacanth gum, gelatin, calcium silicate, cellulose, microcrystalline cellulose, polyvinylpyrrolidone, sterile water, syrup and methylcellulose. The composition may additionally contain lubricants, such as magnesium stearate, talc and mineral oil; wetting agents; emulsifiers and suspending agents; preservatives, such as methyl and propyl hydroxybenzoates; sweeteners; and flavoring agents. After being administered to a patient, the composition of the present invention can provide the effect of rapid, continued or delayed release of the active ingredient.

According to the present invention, the form of said composition may be tablets, pills, powder, lozenges, packets, troches, elixers, suspensions, lotions, solutions, syrups, soft and hard gelatin capsules, suppositories, sterilized injection fluid, and packaged powder. The composition of the present invention may be delivered via any physiologically acceptable route, such as oral and parenteral (e.g., intramuscular, intravenous, subcutaneous, and intraperitoneal). In certain embodiments, the composition of the present invention is administered as a liquid injectable formulation which can be provided as a ready-to-use dosage form or as a reconstitutable stable powder. Preparation of an appropriate parenteral composition under sterile conditions may be accomplished with standard pharmacological techniques well known to persons skilled in the art. In some embodiment, the composition of the present invention is administered orally. Orally acceptable dosage forms include, but are not limited to, tablets, capsules, powder, emulsions and aqueous suspensions. In certain embodiments, commonly used carriers for tablets include lactose and corn starch. Generally, a lubricant such as magnesium stearate can also be added to the tablets. When necessary, some sweeteners, flavors or coloring agents can be added.

In particular embodiments, the composition of the present invention can be used as food or medicine.

According to the present invention, the extracts of Salvia miltiorrhiza and/or Poncirus trifoliata can be used for inhibiting body weight gain of a subject. Specifically, the extracts of Salvia miltiorrhiza and/or Poncirus trifoliata as active ingredients, are administered in amounts effective in inhibiting body weight gain and can be used for weight loss. In some embodiments, an extract of Salvia miltiorrhiza is administered to a subject in need thereof. In some embodiments, an extract of Poncirus trifoliata is administered to a subject in need thereof. In some embodiments, the extract of Salvia miltiorrhiza is administered in amounts in a range of 10 to 1,000 mg/kg/week, such as 10 to 900 mg/kg/week, 10 to 800 mg/kg/week, 10 to 700 mg/kg/week, 10 to 600 mg/kg/week, 10 to 500 mg/kg/week, 10 to 400 mg/kg/week, 10 to 300 mg/kg/week, 10 to 200 mg/kg/week, 10 to 100 mg/kg/week, 10 to 50 mg/kg/week or 10 to 25 mg/kg/week; or 50 to 900 mg/kg/week, 75 to 900 mg/kg/week, 100 to 900 mg/kg/week, 150 to 900 mg/kg/week, 200 to 900 mg/kg/week, 300 to 900 mg/kg/week, 300 to 800 mg/kg/week, 300 to 700 mg/kg/week or 300 to 600 mg/kg/week. In some embodiments, the extract of Poncirus trifoliata is administered in amounts in a range of 10 to 300 mg/kg/week, such as 10 to 200 mg/kg/week, 10 to 100 mg/kg/week, 10 to 90 mg/kg/week, 10 to 80 mg/kg/week, 20 to 80 mg/kg/week, 30 to 80 mg/kg/week, 40 to 80 mg/kg/week or 50 to 80 mg/kg/week.

In some embodiments, a combination of an extract of Salvia miltiorrhiza and an extract of Poncirus trifoliata (S+P) is administered to a subject in need thereof. For administration of such combination, the extract of Salvia miltiorrhiza and the extract of Poncirus trifoliata may be administered in sequence, or administered together as a composition. In some embodiments, the extract of Salvia miltiorrhiza and the extract of Poncirus trifoliata may be mixed to form a composition. In some embodiments, the extract of Salvia miltiorrhiza is combined with the extract of Poncirus trifoliata in a proper ratio, for example, in a range of 10:1 to 1:10 (w/w, gram/gram). In some examples, the ratio of the extract of Salvia miltiorrhiza to the extract of Poncirus trifoliata is 10:1 (S:P, w/w, gram/gram), 9:1 (S:P, w/w, gram/gram), 8:1 (S:P, w/w, gram/gram), 7:1 (S:P, w/w, gram/gram. gram), 6:1 (S:P, w/w, gram/gram. gram), 5:1 (S:P, w/w, gram/gram. gram), 4:1 (S:P, w/w, gram/gram. gram), 3:1 (S:P, w/w, gram/gram. gram), 2:1 (S:P, w/w, gram/gram. gram) or 1:1 (S:P, w/w, gram/gram. gram). In some examples, the ratio of the extract of Salvia miltiorrhiza to the extract of Poncirus trifoliata is 1:10 (S:P, w/w, gram/gram), 1:9 (S:P, w/w, gram/gram), 1:8 (S:P, w/w, gram/gram), 1:7 (S:P, w/w, gram/gram. gram), 1:6 (S:P, w/w, gram/gram. gram), 1:5 (S:P, w/w, gram/gram. gram), 1:4 (S:P, w/w, gram/gram. gram), 1:3 (S:P, w/w, gram/gram. gram), 1:2 (S:P, w/w, gram/gram. gram) or 1:1 (S:P, w/w, gram/gram. gram). In some embodiments, the extract of Salvia miltiorrhiza and the extract of Poncirus trifoliata are administered in amounts effective in inhibiting body weight gain to an extent greater, preferably synergistically, than administration of the extract of Salvia miltiorrhiza or the extract of Poncirus trifoliata alone does. In some embodiments, a combination of the extracts of the present invention can reduce the body weight of the individual who takes it weekly for a period of 3 to 10 weeks or longer (3 weeks, 4 weeks, 5 weeks, 6 weeks, 7 weeks, 8 weeks, 9 weeks or 10 weeks or more) by about 1% to about 10%, compared with the reference body weight of the individual who is not administered with the combination of the extracts of the present invention. In some embodiments, the extract of Salvia miltiorrhiza is administered in amounts in a range of 10 to 1,000 mg/kg/week, such as 10 to 900 mg/kg/week, 10 to 800 mg/kg/week, 10 to 700 mg/kg/week, 10 to 600 mg/kg/week, 10 to 500 mg/kg/week, 10 to 400 mg/kg/week, 10 to 300 mg/kg/week, 10 to 200 mg/kg/week, 10 to 100 mg/kg/week, 10 to 50 mg/kg/week or 10 to 25 mg/kg/week; or 50 to 900 mg/kg/week, 75 to 900 mg/kg/week, 100 to 900 mg/kg/week, 150 to 900 mg/kg/week, 200 to 900 mg/kg/week, 300 to 900 mg/kg/week, 300 to 800 mg/kg/week, 300 to 700 mg/kg/week or 300 to 600 mg/kg/week. In some embodiments, the extract of Poncirus trifoliata is administered in amounts in a range of 10 to 300 mg/kg/week, such as 10 to 200 mg/kg/week, 10 to 100 mg/kg/week, 10 to 90 mg/kg/week, 10 to 80 mg/kg/week, 20 to 80 mg/kg/week, 30 to 80 mg/kg/week, 40 to 80 mg/kg/week or 50 to 80 mg/kg/week. In certain examples, an extract of Salvia miltiorrhiza in combination with an extract of Poncirus trifoliata (S+P) are administered to a subject in need thereof, where the extract of Salvia miltiorrhiza is prepared by extracting Salvia miltiorrhiza by 50% to 95% ethanol and administrated at an amount of 300 to 800 mg/kg/week, and the extract of Poncirus trifoliata is prepared by extracting Poncirus trifoliata by 50% to 95% ethanol and administrated at an amount of 30 to 80 mg/kg/week.

In some embodiments, a combination of an extract of Salvia miltiorrhiza and an extract of Poncirus trifoliata is further combined with an extract of Curcuma longa (S+P+C) for administration. For administration of such combination, the extract of Salvia miltiorrhiza, the extract of Poncirus trifoliata and the extract of Curcuma longa may be administered in sequence, or administered together as a composition. In some embodiments, the extract of Salvia miltiorrhiza, the extract of Poncirus trifoliata and the extract of Curcuma longa may be mixed to form a composition. In some embodiments, the extract of Salvia miltiorrhiza, the extract of Poncirus trifoliata and the extract of Curcuma longa are combined together in a proper ratio, for example, in a range of 1 to 15 (S): 1 to 15 (P): 1 to 15 (C) (S:P:C, w/w/w, gram/gram/gram). In some examples, the ratio of the extract of Salvia miltiorrhiza, the extract of Poncirus trifoliata and the extract of Curcuma longa is 1 to 15 (S): 1 to 10 (P): 1 to 5 (C) (S:P:C, w/w, gram/gram). In some examples, the ratio of the extract of Salvia miltiorrhiza, the extract of Poncirus trifoliata and the extract of Curcuma longa is 1 to 15 (S): 1 to 5 (P): 1 to 3 (C) (S:P:C, w/w, gram/gram). In some examples, the ratio of the extract of Salvia miltiorrhiza, the extract of Poncirus trifoliata and the extract of Curcuma longa is 10 to 15 (S): 1 to 3 (P): 1 (C) (S:P:C, w/w, gram/gram). In some examples, the ratio of the extract of Salvia miltiorrhiza, the extract of Poncirus trifoliata and the extract of Curcuma longa is 12 (S): 1.5 (P): 1 (C) (S:P:C, w/w, gram/gram). In some embodiments, the extract of Salvia miltiorrhiza, the extract of Poncirus trifoliata and the extract of Curcuma longa are administered in amounts effective in inhibiting body weight gain to an extent greater, preferably synergistically, than administration of the extract of Salvia miltiorrhiza, the extract of Poncirus trifoliata or the extract of Curcuma longa alone does, or two of them do. In some embodiments, a combination of the extract(s) of the present invention can reduce the body weight of the individual who takes it weekly for a period of 3 to 14 weeks or longer (3 weeks, 4 weeks, 5 weeks, 6 weeks, 7 weeks, 8 weeks, 9 weeks, 10 weeks, 11 week, 12 week, 13 weeks, 14 weeks or more) by about 1% to about 30%, compared with the reference body weight of the individual who is not administered with the combination of the extracts of the present invention. In some embodiments, the extract of Salvia miltiorrhiza is administered in amounts in a range of 10 to 1,000 mg/kg/week, such as 10 to 900 mg/kg/week, 10 to 800 mg/kg/week, 10 to 700 mg/kg/week, 10 to 600 mg/kg/week, 10 to 500 mg/kg/week, 10 to 400 mg/kg/week, 10 to 300 mg/kg/week, 10 to 200 mg/kg/week, 10 to 100 mg/kg/week, 10 to 50 mg/kg/week or 10 to 25 mg/kg/week; or 50 to 900 mg/kg/week, 75 to 900 mg/kg/week, 100 to 900 mg/kg/week, 150 to 900 mg/kg/week, 200 to 900 mg/kg/week, 300 to 900 mg/kg/week, 300 to 800 mg/kg/week, 300 to 700 mg/kg/week or 300 to 600 mg/kg/week. In some embodiments, the extract of Poncirus trifoliata is administered in amounts in a range of 10 to 300 mg/kg/week, such as 10 to 200 mg/kg/week, 10 to 100 mg/kg/week, 10 to 90 mg/kg/week, 10 to 80 mg/kg/week, 20 to 80 mg/kg/week, 30 to 80 mg/kg/week, 40 to 80 mg/kg/week or 50 to 80 mg/kg/week. In some embodiments, the extract of Curcuma longa is administered in amounts in a range of 10 to 150 mg/kg/week, such as 10 to 100 mg/kg/week, 10 to 90 mg/kg/week, 10 to 80 mg/kg/week, 10 to 70 mg/kg/week, 10 to 60 mg/kg/week, 20 to 60 mg/kg/week, or 30 to 60 mg/kg/week mg/kg/week. In certain examples, an extract of Salvia miltiorrhiza plus an extract of Poncirus trifoliata in combination with an extract of Curcuma longa (S+P+C) are administered to a subject in need thereof, where the extract of Salvia miltiorrhiza is prepared by extracting Salvia miltiorrhiza by 50% to 95% ethanol and administrated at an amount of 300 to 800 mg/kg/week, the extract of Poncirus trifoliata is prepared by extracting Poncirus trifoliata by 50% to 95% ethanol and administrated at an amount of 30 to 80 mg/kg/week, and the extract of Curcuma longa is prepared by extracting Curcuma longa by 90% to 95% ethanol and administrated at an amount of 30 to 80 mg/kg/week.

The World Health Organization uses the Body Mass Index (BMI) to measure the degree of obesity. The calculation formula is weight (kg) divided by the square of height (meters). According to the definition of the World Health Organization, normal individuals have a BMI value of 18.5 to 24.9, a BMI value greater than 25.0 is considered overweight, and a BMI value greater than 30.0 is considered obese. Among them, a BMI value of 30-34.9 is the first degree of obesity, and a BMI value of 35-39.9 is the second degree of obesity, and a BMI value of 40 or more is the third degree of obesity.

In some embodiments, the method of the present invention is performed for cosmetic purpose for improving body appearance. In some embodiments, the method of the present invention is performed for therapeutic purpose for treating a disease, such as obesity.

In some embodiments, the extract(s) of the present invention is administered to normal individuals for preventing overweight or obesity. In some embodiments, the extract of the present invention is administered to individuals who are overweight or obese. By slowing down or inhibiting weight gain, the desired therapeutic effect (for example, lowering BMI to arrive at a normal value) can be achieved.

In some embodiment, the extract(s) of the present invention is effective in treating a disease relevant to overweight or obese, including diabetes, hyperlipidemia, arteriosclerosis, and hypertension.

According to the present invention, the extracts of Salvia miltiorrhiza and/or Poncirus trifoliata optionally in combination with the extract of Curcuma longa can be administrated as described herein for improving liver function. In some embodiments, the extract of Salvia miltiorrhiza and/or the extract of Poncirus trifoliata optionally in combination with an extract of Curcuma longa as described herein is/are administrated in amounts effective in improving liver function of the subject. In some embodiments, the extract of Salvia miltiorrhiza and/or the extract of Poncirus trifoliata optionally in combination with an extract of Curcuma longa as described herein is/are administrated in amounts effective in reducing glutamic oxaloacetic transaminase (GOT) levels and/or glutamic pyruvic transaminase (GPT) levels in the subject. In some embodiments, the extract of Salvia miltiorrhiza and the extract of Poncirus trifoliata is combined with the extract of Curcuma longa (S+P+C) for administration. Specifically, the extract of Salvia miltiorrhiza, the extract of Poncirus trifoliata and the extract of Curcuma longa are administered in amounts effective in improving liver function to an extent greater, preferably synergistically, than administration of the extract of Salvia miltiorrhiza, the extract of Poncirus trifoliata or the extract of Curcuma longa alone does, or two of them do.

The present invention also provides a kit comprising (i) an extract of Salvia miltiorrhiza; and/or (ii) an extract of Poncirus trifoliata, and optionally (iii) an extract of Curcuma longa. The kit of the present invention is useful for inhibiting body weight gain. The kit of the present invention is also useful for improving liver function. Such kits may include one or more containers comprising the extract of Salvia miltiorrhiza and/or the extract of Poncirus trifoliata and optionally the extract of Curcuma longa. In some embodiments, the kit may comprise instructions for use in accordance with any of the methods described herein. The instructions relating to the use of the extract(s) as described herein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert. The kits of this invention are in suitable packaging. Suitable packaging includes, but is not limited to, vials, bottles, jars, flexible packaging, and the like.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

1. Material and Methods

1.1 Plant materials

Salvia miltiorrhiza (dried root), Poncirus trifoliata (dried fruit), Curcuma longa (fresh rhizome) were purchased from a traditional Chinese medicine store in Taipei, Taiwan.

1.2 Preparation of Extract Products

1.2.1 Supercritical Carbon Dioxide Extraction of Salvia Miltiorrhiza

About 100±30 g of Salvia miltiorrhiza (dried roots) was subjected to static carbon dioxide extraction at a temperature ranging from 35 to 80° C. with a CO₂ pressure ranging from 20 to 50 MPa for 0.5 to 2 hours, and then the extract was collected for 5 to 15 minutes. The above cycle was repeated until the weight of the extract was constant. Total extraction time was about 2 to 12 hours. The final product is about 0.5 to 1.5 g of red viscous paste with low liquidity.

1.2.2 Ethanol Extraction of Salvia Miltiorrhiza

About 20 to 30 g of Salvia miltiorrhiza (dried roots) was added with 100 to 600 mL of 50% to 95% ethanol (solid-to-liquid ratio being 1:5 to 1:20) and was ultrasonically extracted (frequency: 28-40 kHz, power: 400-600 W) at a temperature ranging from 25 to 50° C. for 30 to 120 minutes, centrifuged at 5,000-9,000 rpm for 5 to 15 minutes, and then filtered with a filter paper. The above process was repeated twice. Filtrates were combined and concentrated to dryness, and then refrigerated for at least 8 hours to completely frozen. The extract was freeze-dried and resulted in 0.25 to 0.6 g of red solid.

1.2.3 Ethanol Extraction of Poncirus trifoliata

About 20 to 30 g of Poncirus trifoliata (dried fruits) was added with 100 to 600 mL of 50% to 95% ethanol (solid-to-liquid ratio being 1:5 to 1:20) and was ultrasonically extracted (frequency: 28-40 kHz, power: 400-600 W) at a temperature ranging from 25 to 50° C. for 30 to 120 minutes, centrifuged at 5,000-9,000 rpm for 30 to 12 minutes, and then filtered with a filter paper. The above process was repeated twice. Filtrates were combined and concentrated to dryness, and then refrigerated for at least 8 hours to completely frozen. The extract was freeze-dried and resulted in 1.5-3.0 g of dark green solid.

1.2.4 Ethanol Extraction of Curcuma longa

The rhizomes of fresh red turmeric were washed with deionized water and then freeze-dried for 24 hours. Then, it was ground with a pulverizer, and then screened with a sieve with a pore size of 0.18 mm. The powder of Curcuma longa rhizome (30 g) was extracted using 150 mL ethanol (90% or more) at a rotating speed of 100 to 300 rpm for about 30 minutes to obtain an ethanol-extracted product. The ethanol-extracted product is subjected to crystallization of curcuminoids at a temperature of 2 to 8° C. and a rotation speed of 40 to 300 rpm using ethanol (90% or more).

1.3 Cells and Oil Red 0 Staining

3T3-L1 cells were maintained in Dulbecco's modified Eagle's medium supplemented with 10% calf serum, 100 U/ml penicillin, and 0.1 mg/ml streptomycin. For differentiation into adipocytes, the cells were cultured in the differentiation medium containing 5 μg/ml insulin, 0.5 mM 3-isobutyl-1-methylxanthine, 1 μM dexamethasone. In the first experiment, the cells were cultured in the differentiation medium added with the supercritical carbon dioxide extract of Salvia miltiorrhiza (SME-CO₂, 1.2 μg/ml), the ethanol extract of Poncirus trifoliata (PTE-EtOH, 10 μg/ml), a combination of SME-CO₂ (1.2 μg/ml) and PTE-EtOH (10 μg/ml), and 0.5% DMSO (control group), respectively. In the second experiment, the cells were cultured in the differentiation medium added with the ethanol extract of Salvia miltiorrhiza (SME-EtOH, 25 μg/ml), PTE-EtOH (10 μg/ml), a combination of SME-EtOH (25 μg/ml) and PTE-EtOH (10 μg/ml), and 0.5% DMSO (control group), respectively. In the third experiment, the cells were cultured in the differentiation medium added with SME-EtOH (5 μg/ml), PTE-EtOH (5 μg/ml), the ethanol extract of Curcuma longa (CLE-EtOH, 1 μg/ml), a combination of SME-EtOH (5 μg/ml) and PTE-EtOH (5 μg/ml), a combination of SME-EtOH (5 μg/ml) and CLE-EtOH (1 μg/ml), a combination of SME-EtOH (5 μg/ml), PTE-EtOH (5 μg/ml) and CLE-EtOH (1 μg/ml), and 0.5% DMSO (control group). The extract (paste or solid) was first dissolved in 100% DMSO to prepare a sample of 25 mg/ml which was then added to the culture medium to reach a concentration as needed in the cell culture as described above. After culturing for 8 days, the cells were washed, fixed and stained with Oil Red 0 solution to observe and quantify the lipid droplet formation. The stained Oil Red 0 was eluted with isopropanol and the optical absorbance was measured at 500 nm for quantification.

1.4 Animal Treatment

Eight-week-old C57BL/6 mice were given normal diet (normal control). After 4 weeks, the mice were fed with high fat diet (45% of calories from fat), and grouped and administered through intraperitoneal injection with different extracts. In the first experiment, the mice were administered with SME-EtOH (600 mg/kg/week), SME-CO₂ (15 mg/kg/week), PTE-EtOH (75 mg/kg/week), and 100% DMSO (control group), respectively, for 6 weeks. In the second experiment, the mice were administered with SME-EtOH (300 mg/kg/week), PTE-EtOH (50 mg/kg/week), SME-EtOH (300 mg/kg/week) plus PTE-EtOH (50 mg/kg/week) and 100% DMSO (control group), respectively, for 7 weeks. In the third experiment, the mice were administered with SME-CO₂ (5 mg/kg/week), PTE-EtOH (50 mg/kg/week), SME-CO₂ (5 mg/kg/week) plus PTE-EtOH (50 mg/kg/week), and 100% DMSO (control group), respectively, for 7 weeks. The mice's body weights were measured and recorded weekly. In the oral administration experiment, eight-week-old C57BL/6 mice were given normal diet. After 4 weeks, the mice were fed with high fat diet (45% of calories from fat), and grouped and administered through oral with different extracts. Mice were oral administered with SME-EtOH (600 mg/kg/week), SME-EtOH (600 mg/kg/week) plus PTE-EtOH (75 mg/kg/week), SME-EtOH (600 mg/kg/week) plus PTE-EtOH (75 mg/kg/week) combined with CLE-EtOH (50 mg/kg/week) and 100% DMSO (control group), respectively, for 14 weeks.

1.5 Measurement of Biochemical Parameters

Blood samples (500 μL) were collected from the tail vein and centrifuged (6000 g for 3 min) to separate serum from cells. Serum biochemical parameters were measured within 24 h. The levels of serum blood urea nitrogen (BUN), creatinine (CRE), glucose, triglycerides (TG), glutamic oxaloacetic transaminase (GOT), and glutamate pyruvate transaminase (GPT) were measured using a Spotchem EZ SP 4430 analyzer.

1.6 Statistical Analysis

Prism software was used for difference analysis (t-test and two-way ANOVA).

2. Results

2.1 Extracts of Salvia miltiorrhiza and/or Poncirus trifoliata can Inhibit Lipid Droplet Formation in Adipocytes

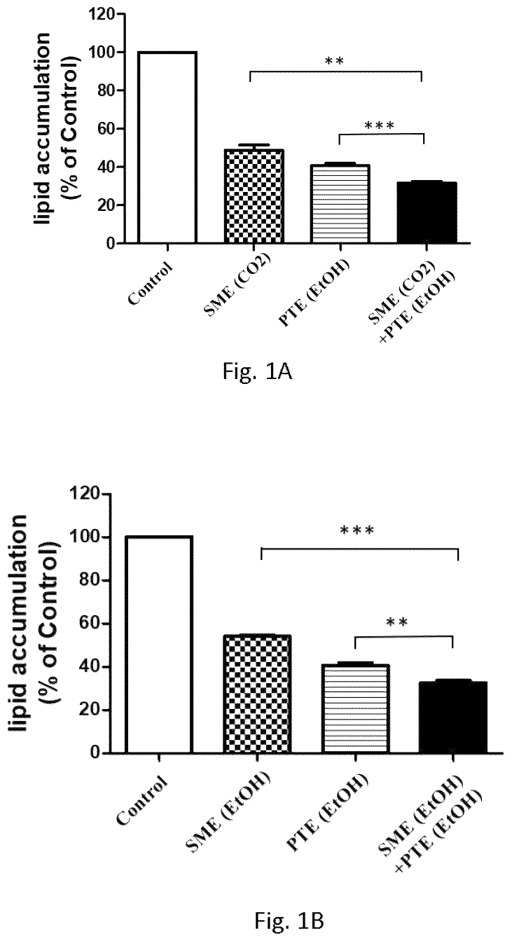

The experiments of Oil Ref 0 staining were conducted with 3T3-L1 cells. The results show that each of SME-CO₂ (1.2 μg/ml), SME-EtOH (25 μg/ml) and PTE-EtOH (10 Kg/ml) significantly inhibited the formation of lipid droplets in the adipocytes. The average lipid droplet formation was reduced by 51% with SME-CO₂ (1.2 μg/ml), 46% with SME-EtOH (25 μg/ml) and 59% with PTE-EtOH (10 μg/ml), as compared to the control group (FIG. 1A and FIG. 1B). The results further show that a combination of Salvia miltiorrhiza and Poncirus trifoliata, including SME-CO₂ (1.2 μg/ml) plus PTE-EtOH (10 μg/ml) and SME-EtOH (25 μg/ml) plus PTE-EtOH (10 μg/ml), exhibited a more significant inhibitory effect on lipid droplet formation in adipocytes. The average lipid droplet formation was reduced by about 68% with SME-CO₂ plus PTE-EtOH (FIG. 1A) and 67% with SME-EtOH plus PTE-EtOH (FIG. 1B), respectively, as compared to the control group.

2.2 Extracts of Salvia miltiorrhiza and/or Poncirus trifoliata can Inhibit Body Weight Gain in Animals

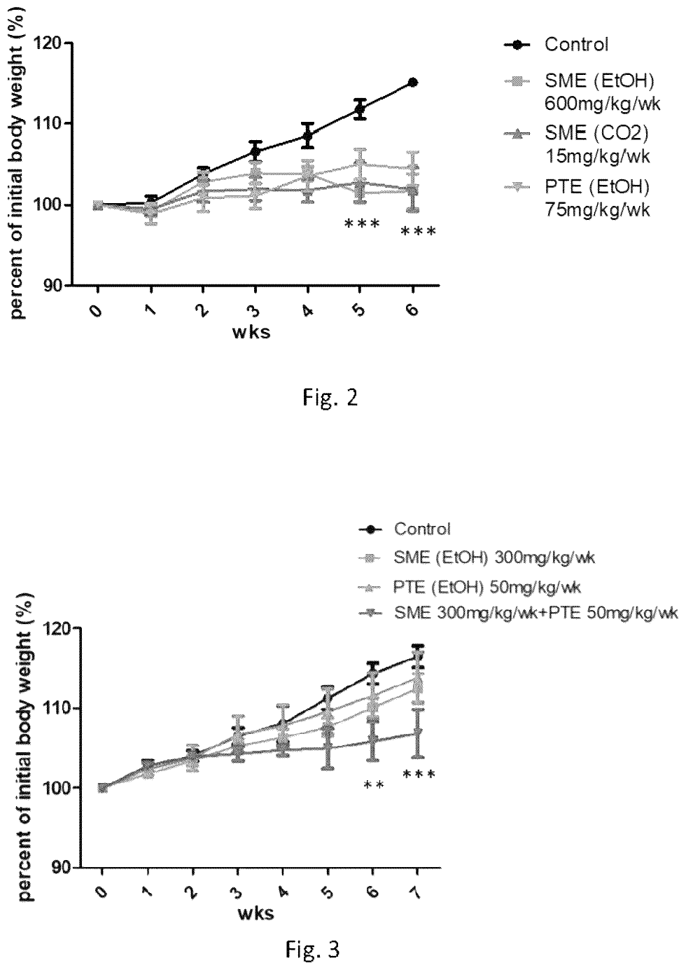

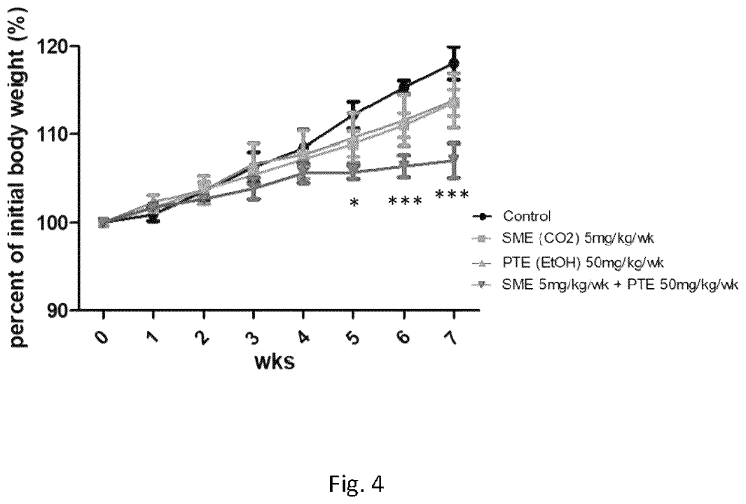

In addition to cell experiments, an animal model of high-fat diet (HFD) induced obesity was set up to examine the effects of the extracts of Salvia miltiorrhiza and/or Poncirus trifoliata on inhibiting obesity. The results show that weight gain of HFD-induced obese mice administered with SME-CO₂ (15 mg/kg/week), SME-EtOH (600 mg/kg/week) or PTE-EtOH (75 mg/kg/week) was slower than that of the control group over 6 weeks, indicating that the extracts of the present invention each effectively suppressed weight gain. See FIG. 2. When SME-EtOH (300 mg/kg/week) or SME-CO₂ (5 mg/kg/week) was administered together with PTE-EtOH (50 mg/kg/week), the weight gain of HFD-induced obese mice was further suppressed as compared to that of the mice administered with SME-CO₂ (5 mg/kg/week), SME-EtOH (300 mg/kg/week) or PTE-EtOH (50 mg/kg/week). See FIG. 3 and FIG. 4.

2.3 Extracts of Salvia miltiorrhiza Plus Poncirus trifoliata Combined Curcuma longa can Inhibit Lipid Droplet Formation in Adipocytes

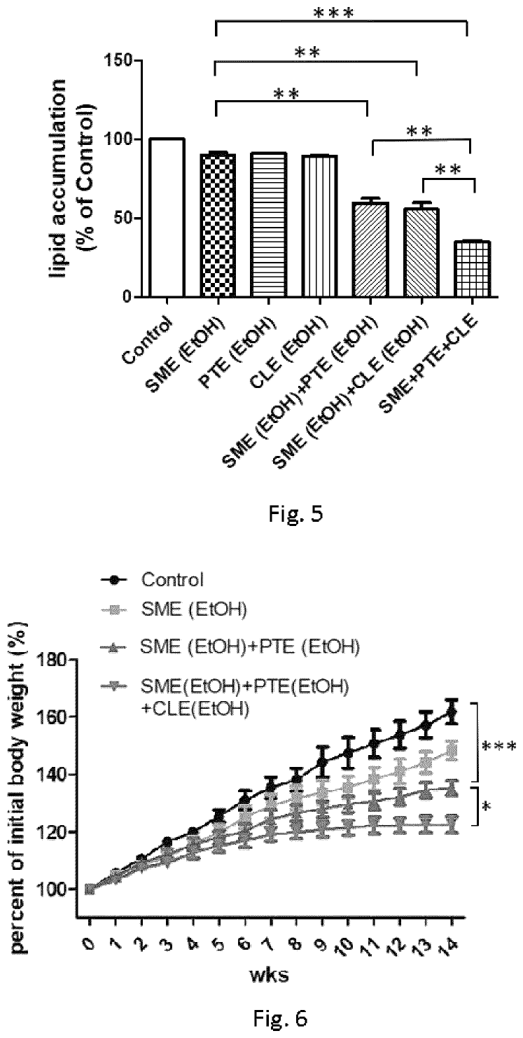

The experiments of Oil Ref 0 staining were conducted with 3T3-L1 cells. The results show that each of SME-EtOH (5 ng/ml) plus PTE-EtOH (5 μg/ml), SME-EtOH (5 μg/ml) plus CLE-EtOH (1 μg/ml), and SME-EtOH (5 μg/ml) plus PTE-EtOH (5 μg/ml) with CLE-EtOH (1 μg/ml) significantly inhibited the formation of lipid droplets in the adipocytes as compared to individual SME-EtOH (5 μg/ml), PTE-EtOH (5 μg/ml) and CLE-EtOH (1 μg/ml). The average lipid droplet formation was reduced by 40% with SME-EtOH (5 ng/ml) plus PTE-EtOH (5 μg/ml), 44% with SME-EtOH (5 μg/ml) plus CLE-EtOH (1 μg/ml) and 65% with SME-EtOH plus PTE-EtOH combined with CLE-EtOH, as compared to the control group (FIG. 5). The results further show that a combination of Salvia miltiorrhiza (SME-EtOH), Poncirus trifoliata (PTE-EtOH) and Curcuma longa (CLE-EtOH) exhibited a more significant inhibitory effect on lipid droplet formation in adipocytes.

2.4 Extracts of Salvia miltiorrhiza Plus Poncirus trifoliata Combined with Curcuma longa can Inhibit Body Weight Gain in Animals

In the oral administered animal model, high-fat diet (HFD) induced obesity was set up to examine the effects of the extracts of Salvia miltiorrhiza, Poncirus trifoliata and Curcuma longa on inhibiting obesity. The results show that weight gain of HFD-induced obese mice administered with SME-EtOH (600 mg/kg/week), SME-EtOH (600 mg/kg/week) plus PTE-EtOH (75 mg/kg/week), and SME-EtOH (600 mg/kg/week) plus PTE-EtOH (75 mg/kg/week) combined with CLE-EtOH (50 mg/kg/week) were slower than that of the control group over 14 weeks, effectively suppressing weight gain. Especially, when Salvia miltiorrhiza (SME-EtOH) was administered together with Poncirus trifoliata (PTE-EtOH) and Curcuma longa (CLE-EtOH), the weight gain of HFD-induced obese mice was further suppressed as compared to that of the mice administered with SME-EtOH only and a combination of SME-EtOH and PTE-EtOH, respectively (FIG. 6).

2.5 Extracts of Salvia miltiorrhiza Plus Poncirus trifoliata Combined Curcuma longa can Inhibit Biochemical Parameters Related to Liver Function in Animals

After 14 weeks of oral administration of various extracts after 4-week HFD supply, the investigation of serum hepatic biochemical parameters demonstrated that SME-EtOH (600 mg/kg/week) plus PTE-EtOH (75 mg/kg/week) and SME-EtOH (600 mg/kg/week) plus PTE-EtOH (75 mg/kg/week) combined with CLE-EtOH (50 mg/kg/week) depressed the concentration of glutamic oxaloacetic transaminase (GOT) and glutamate pyruvate transaminase (GPT) in mice as compared to HFD control group (Table 2).

TABLE 2
SME-EtOH plus PTE-EtOH and SME-EtOH plus PTE-EtOH
combined with CLE-EtOH can improve liver function.

| | Biochemical parameters | | | | | | |
|---|---|---|---|---|---|---|---|
| | Glu | TG | Cho | GOT | GPT | BUN | Cre |
| Groups | (mg/dL) | (mg/dL) | (mg/dL) | (IU/L) | (IU/L) | (mg/dL) | (mg/dL) |
| Normal | 87.3 ± 3.9 | 99.3 ± 14 | 68.8 ± 9.6 | 66.2 ± 11.5 | 31.6 ± 4.4 | 25.75 ± 4.8 | 0.2 |
| Control | | | | | | | |
| HFD Control | 170 ± 32 | 100 ± 11 | 220 ± 19 | 113 ± 43 | 115 ± 55 | 30 ± 3.0 | 0.2 |
| SME-EtOH | 180 ± 39 | 108 ± 19 | 202 ± 47 | 94 ± 47 | 72 ± 35 | 31 ± 3 | 0.2 |
| SME-EtOH + | 166 ± 25 | 102 ± 14 | 191 ± 23 | 61 ± 28 | 43 ± 37 | 32 ± 3 | 0.2 |
| PTE-EtOH | | | | | | | |
| SME-EtOH + | 164 ± 12 | 101 ± 39 | 180 ± 10 | 55 ± 10 | 40 ± 8 | 28 ± 2 | 0.2 |
| PTE-EtOH + | | | | | | | |
| CLE-EtOH | | | | | | | |

Our results show that SME-EtOH plus PTE-EtOH and SME-EtOH plus PTE-EtOH combined with CLE-EtOH can improve liver function.

3. Conclusion

The results of the above cell experiments and animal study show that extracts of Salvia miltiorrhiza and/or Poncirus trifoliata, optionally with Curcuma longa can inhibit lipid droplet formation in adipocytes and body weight gain in animals. In particular, an extract of Salvia miltiorrhiza in combination with an extract of Poncirus trifoliata and/or Curcuma longa exhibits a more significant inhibitory effect on cellular lipid droplet formation and weight gain of mice, and improved liver function, as compared to that achieved by a single extract product. Moreover, a combination of Salvia miltiorrhiza (SME-EtOH), Poncirus trifoliata (PTE-EtOH) and Curcuma longa (CLE-EtOH) exhibited a more significant inhibitory effect on body weight loss compared to Salvia miltiorrhiza (SME-EtOH) plus Poncirus trifoliata (PTE-EtOH). It is demonstrated that an extract of Salvia miltiorrhiza and/or an extract of Poncirus trifoliata optionally in combination with an extract of Curcuma longa are useful in fat loss or weight loss and prevention and treatment of obesity-induced metabolic syndrome.

What is claimed is:

1. A method for inhibiting body weight gain in a subject in need thereof, comprising administering to the subject an effective amount of an extract of Salvia miltiorrhiza and/or an extract of Poncirus trifoliata, and optionally an extract of Curcuma longa.

2. The method of claim 1, wherein the extract of Salvia miltiorrhiza is prepared by extracting Salvia miltiorrhiza with a first solvent, the extract of Poncirus trifoliata is prepared by extracting Poncirus trifoliata with a second solvent, and/or the extract of Curcuma longa is prepared by extracting Curcuma longa with a third solvent.

3. The method of claim 1, wherein the extract of Salvia miltiorrhiza, the extract of Poncirus trifoliata, and/or the extract of Curcuma longa is/are obtained by supercritical fluid extraction.

4. The method of claim 2, wherein the first solvent, the second solvent and/or the third solvent is/are an alcohol.

5. The method of claim 4, wherein the alcohol is selected from the group consisting of methanol, ethanol, propanol and butanol.

6. The method of claim 3, wherein the supercritical fluid extraction process utilizes carbon dioxide as solvent.

7. The method of claim 1, wherein the extract of Salvia miltiorrhiza and/or the extract of Poncirus trifoliata, and optionally the extract of Curcuma longa, is/are administrated in an amount effective in inhibiting lipid accumulation in adipocytes.

8. The method of claim 1, wherein the extract of Salvia miltiorrhiza and/or the extract of Poncirus trifoliata, and optionally the extract of Curcuma longa is/are administrated in an amount effective in treating or avoiding obesity of the subject.

9. The method of claim 1, comprising administering the extract of Salvia miltiorrhiza and the extract of Poncirus trifoliata to the subject.

10. The method of claim 9, wherein the extract of Salvia miltiorrhiza and the extract of Poncirus trifoliata are administered in amounts effective in inhibiting body weight gain in the subject to an extent greater than administration of the extract of Salvia miltiorrhiza or the extract of Poncirus trifoliata alone does.

11. The method of claim 1, comprising administering the extract of Salvia miltiorrhiza, the extract of Poncirus trifoliata and the extract of Curcuma longa to the subject.

12. The method of claim 1, wherein the extract of Salvia miltiorrhiza and/or the extract of Poncirus trifoliata, and optionally the extract of Curcuma longa, is/are administrated in amounts effective in improving liver function of the subject.

13. The method of claim 12, wherein the extract of Salvia miltiorrhiza and/or the extract of Poncirus trifoliata, and optionally the extract of Curcuma longa, is/are administrated in amounts effective in reducing glutamic oxaloacetic transaminase (GOT) levels and/or glutamic pyruvic transaminase (GPT) levels in the subject.

14. A composition comprising an extract of Salvia miltiorrhiza and/or an extract of Poncirus trifoliata, and optionally an extract of Curcuma longa, and a physiologically acceptable carrier, wherein the extract of Salvia miltiorrhiza and/or the extract of Poncirus trifoliata, and optionally the extract of Curcuma longa are present in amounts effective in inhibiting body weight gain in a subject in need thereof.

15. The composition of claim 14, wherein the extract of Salvia miltiorrhiza is prepared by extracting Salvia miltiorrhiza with a first solvent, the extract of Poncirus trifoliata is prepared by extracting Poncirus trifoliata with a second solvent and/or the extract of Curcuma longa is prepared by extracting Curcuma longa with a third solvent.

16. The composition of claim 14, wherein the extract of Salvia miltiorrhiza, the extract of Poncirus trifoliata, and/or the extract of Curcuma longa is/are obtained by supercritical fluid extraction.

17. The composition of claim 15, wherein the first solvent, the second solvent and/or the third solvent is/are an alcohol.

18. The composition of claim 17, wherein the alcohol is selected from the group consisting of methanol, ethanol, propanol and butanol.

19. The composition of claim 16, wherein the supercritical fluid extraction process utilizes carbon dioxide as solvent.

20. The composition of claim 11, wherein the extract of Salvia miltiorrhiza and/or the extract of Poncirus trifoliata, and optionally the extract of Curcuma longa is/are present in an amount effective in inhibiting lipid accumulation in adipocytes.

21. The composition of claim 11, wherein the extract of Salvia miltiorrhiza and/or the extract of Poncirus trifoliata, and optionally the extract of Curcuma longa is/are present in an amount effective in treating or avoiding obesity of the subject.

22. The composition of claim 11, wherein the composition comprises the extract of Salvia miltiorrhiza and the extract of Poncirus trifoliata.

23. The composition of claim 22, wherein the extract of Salvia miltiorrhiza and the extract of Poncirus trifoliata are present in amounts effective in inhibiting body weight gain in the subject to an extent greater than administration of the extract of Salvia miltiorrhiza or the extract of Poncirus trifoliata alone does.

24. The composition of claim 14, wherein the composition comprises the extract of Salvia miltiorrhiza, the extract of Poncirus trifoliata and the extract of Curcuma longa.

25. The composition of claim 14, wherein the extract of Salvia miltiorrhiza and/or the extract of Poncirus trifoliata, and optionally the extract of Curcuma longa is/are present in amounts effective in improving liver function of the subject.

26. The composition of claim 25, wherein the extract of Salvia miltiorrhiza and/or the extract of Poncirus trifoliata optionally in combination with an extract of Curcuma longa is/are present in amounts effective in reducing glutamic oxaloacetic transaminase (GOT) levels and/or glutamic pyruvic transaminase (GPT) levels in the subject.

27. A kit comprising
an extract of Salvia miltiorrhiza; and/or
an extract of Poncirus trifoliata; and optionally
an extract of Curcuma longa.
